(19) United States
(12) Patent Application Publication
(10) Pub. No.: US 2008/0188833 A1
(43) Pub. Date: Aug. 7, 2008

(54) Suction cannula

(76) Inventor: Ahmmed Ziah Taufig, Cologne (DE)

Correspondence Address: Vincent L. Ramik, Suite 101, 7345 McWhorter Place, Annandale, VA, 22003, US

(21) Appl. No.: 11/700,852

(22) Filed: Feb. 1, 2007

(51) Int. Cl. A61M 1/00

(52) U.S. Cl. 604/540

(57) ABSTRACT

The invention relates to a suction cannula (10) for sucking biological tissue from a human body, comprising a suction tube (12) with a suction aperture (20), the suction tube (12) further comprising a cleaning aperture (22). Further, a cleaning aperture closure (38) is provided which, in its closing state, closes the cleaning aperture and, in its opening state, releases the cleaning aperture (22).

The invention relates to a suction cannula with a suction tube and at least one suction aperture for sucking biological tissue from a living human body.

In the form of suction cannulas, e.g., suction cannulas serve to suck body tissue or fat and to this end, they comprise a suction tube and at least one suction aperture at the distal end of the suction tube through which the body fat can be sucked from the body interior. Those cannulas normally employed for liposuction are reusable cannulas made of stainless steel. After a liposuction, the cannula has to be cleaned and sterilized for the next treatment. To this end, the interior and the exterior of the cannula have to be cleaned mechanically first. The closed cannula interior can only be cleaned mechanically by introducing a washing liquid. A true mechanical cleaning of the cannula interior cannot be done without relatively great efforts. In any case, a visual check of the cleaning success is excluded.

The subsequent vapor sterilization bears the risk that the cannula interior is not sufficiently sterilized because of its poor accessability.

Most complications in liposuction are due to infections. To a large part, non-sterile suction cannulas are made responsible therefore.

It is the object of the present invention to provide a suction cannula that is better able to be sterilized.

This object is solved, according to the invention, by a suction cannula with the features of claim 1.

In the suction cannula according to the invention, the suction tube comprises at least one cleaning aperture and a cleaning aperture closure closing the cleaning aperture in its closing state and releasing the cleaning aperture in its opening state. This means that the suction cannula does not only have one or more suction apertures, preferably at its distal end, but also one or more cleaning apertures between the distal end of the suction tube and the proximal end of the suction tube, i.e., the end which is connected to a suction apparatus. Access to the interior of the suction tube is improved by the cleaning aperture. The cleaning aperture can be provided in a region of the suction tube the cleaning or vapor-sterilization of which is extremely difficult, as experience shows. This region may be located, for example, approximately halfway of the length of the suction tube. The sterilization vapor may now also enter through the cleaning aperture so that the suction tube can also be sterilized well in its central portion.

As to the shape and cross section of the aperture, the cleaning aperture may be adapted to a mechanical cleaning instrument so that the mechanical cleaning of the suction tube interior is made easier thereby as well.

The cleaning aperture may be closed again by means of the cleaning aperture closure prior to the next application of the cannula for sucking off fat so that during a liposuction, no sucking via the cleaning aperture will take place.

Preferably, the cleaning aperture closure is formed by a closure tube that, in its closing state, is seated on the outside of the suction tube and covers the cleaning aperture. In the opening state, the closure tube may be axially withdrawn from the suction tube or rather twisted or displaced in circumferential or longitudinal direction with respect to the suction tube to release the one or more cleaning apertures in this manner by aligning them with corresponding apertures of the closure tube.

According to a preferred embodiment, the closure tube is an expendable article that is not reused but disposed of after being used once. Thereby, the sterilization of the closure tube for the following application is dropped. By configuring the closure tube as an expendable article, the closure tube can be constructed exclusively with respect to its actual functions, i.e., the simple closing of the cleaning aperture, a simple manipulation and possible further functions. The closure tube configured as an expendable article does not have to be constructed with respect to its ability of being repeatedly sterilized and particularly not with respect to being simply mechanically cleaned from inside. Therefore, the disposable closure tube does not have a separate cleaning aperture. In contrast to the closure tube, the suction tube, which normally is much more complex, is reusable.

Preferably, the suction tube is made of steel and the closure tube is made of plastic. The plastic suction tube can be produced at low costs in an injection molding process but may nevertheless have complex structures, is easily sterilizable for a single application and may have favorable mechanical properties, particularly a certain resiliency which simplifies or facilitates the mounting and fixing of the closure tube to the suction tube.

The disposable suction tube may be mounted to the closure tube by a female thread, for example, which is screwed onto a corresponding male thread of the suction tube. Alternatively, the closure tube may be provided with annular grooves at its inside which interlock with corresponding outward annular ridges of the suction tube in order to fix the closure tube in axial and, if necessary, peripheral direction.

The closure tube may also be fixed to the suction tube by appropriate clamping or hinged levers or by a union nut.

According to a preferred embodiment, the cleaning aperture is formed by a longitudinal slot in the suction tube. A single longitudinal slot may be provided over almost the entire length of the suction tube. To maintain the stability of the suction tube, it is also possible to axially arrange several longitudinal slots in series or such that they axially overlap and are radially offset to each other. The longitudinal slot may extend to the distal and proximal ends of the suction tube.

Preferably, the closure tube also comprises at least one cleaning aperture. It is true that the closure tube may be configured as a disposable article that is removed and disposed of after a liposuction and is pushed onto the suction tube in the form of a new sterile disposable article for closing the one or more cleaning apertures after the suction tube has been sterilized. If, however, the closure tube is repeatedly used, it must be cleaned and sterilized as well. Since, in this case, the closure tube is not substantially shorter than the suction tube, one is confronted with basically the same problems with respect to cleaning and disinfection as with the suction tube. Therefore, the closure tube also preferably comprises one or several cleaning apertures by which the mechanical cleaning and the vapor-sterilization are made easier and improved. The closure tube cleaning aperture may also be configured as a longitudinal slot, e.g., as a single longitudinal slot over the entire length of the closure tube. In this case, the closure tube is slightly resilient in radial direction and can be put onto the suction tube with a slight bias so that it tightly clamps the latter and closes the one or more cleaning apertures in a liquid-tight manner.

According to a preferred embodiment, the one or more suction apertures are provided in the closure tube. If the closure tube is of approximately the same length as the suction tube, both the suction tube and the closure tube have suction apertures at their distal ends, which are in mutual alignment when the closure tube is closed, i.e., are radially continuously open. The suction apertures may also be provided exclusively at the closure tube.

Preferably, the opening angle of the cleaning aperture amounts to at least 30° in a radial plane. Since suction tubes have a small outer diameter of a few millimeters, the opening angle of the cleaning slot must be sufficiently large to provide a sufficiently large access to the suction tube interior for the mechanical cleaning in particular. The opening angle should be as large as possible but so small that the stability of the suction tube and the sealing in the closing state are not significantly worsened.

Preferably, the suction tube and the closure tube have cooperating fastening elements allocated thereto by means of which the suction tube is fixed at the closure tube in the closing state. Such fastening elements can be formed by the mechanical components of a bayonet closure but also by a male thread at the suction tube and a female thread at the closure tube. At a handle allocated to the suction tube, a female thread may be provided which cooperates with a male thread of the closure tube. Simple guides only representing a protection against twisting but not actually fixing the closure tube with respect to the suction tube in axial direction may also serve as fastening elements. The closure tube may be of approximately the same axial length as the suction tube and be closed at its distal end. Then, the closure tube encompasses the suction tube completely.

Preferably, the cleaning aperture is arranged proximally and separately from the suction aperture. In the region of the suction apertures, a sufficient mechanical cleaning and sterilizability is guaranteed even without cleaning apertures.

In a preferred embodiment, the closure tube has a transparent window or has a completely transparent configuration. The doctor in attendance is able to look at the interior of the suction tube through the closure tube and the cleaning aperture of the suction tube and may thus observe the suction process.

Hereinafter, two embodiments of the invention are explained in detail with reference to the drawings.

In the Figures:

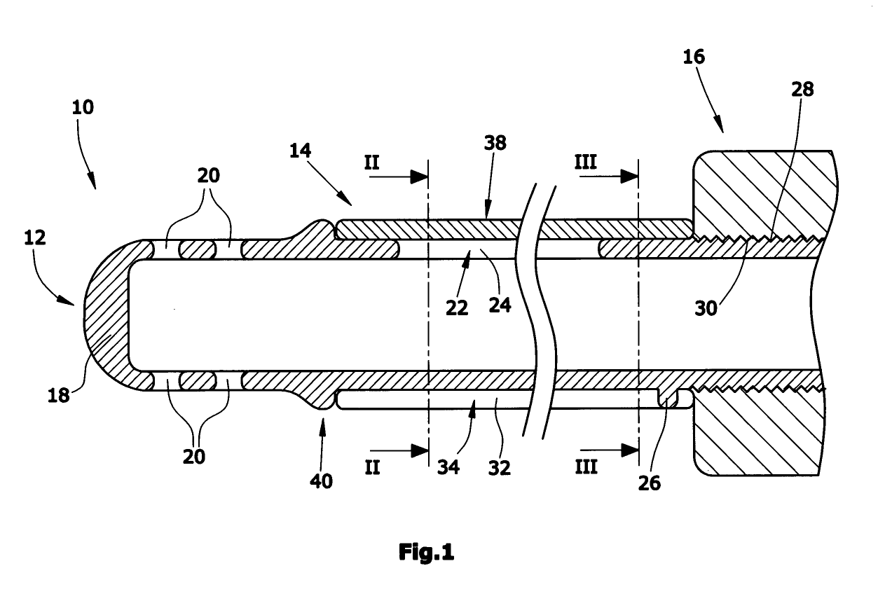

FIG. 1 is a longitudinal section of a suction cannula in the form of a liposuction cannula with a suction tube and a closure tube,

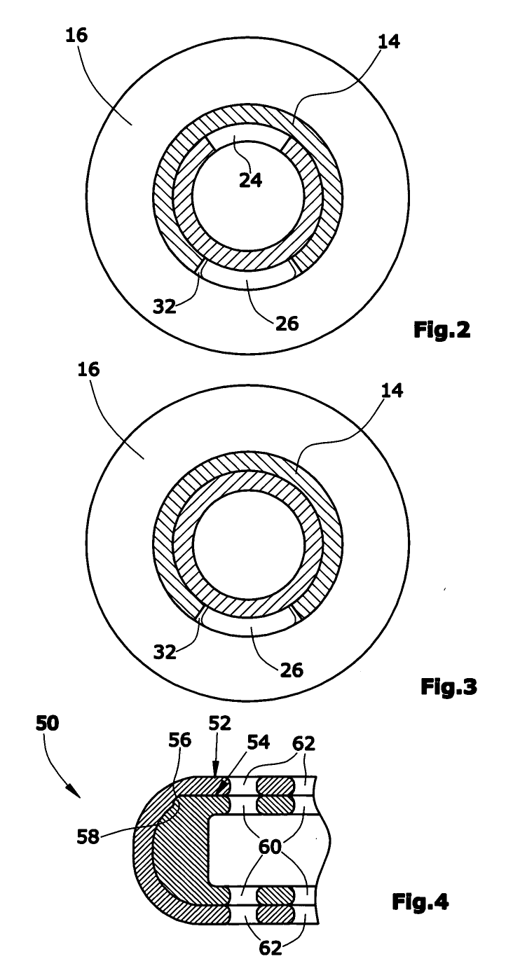

FIG. 2 is a first cross section along the line II-II of the suction cannula of FIG. 1,

FIG. 3 is a cross section in the plane III-III of the suction cannula of FIG. 1,

FIG. 4 is a second embodiment of a suction cannula in longitudinal section, and

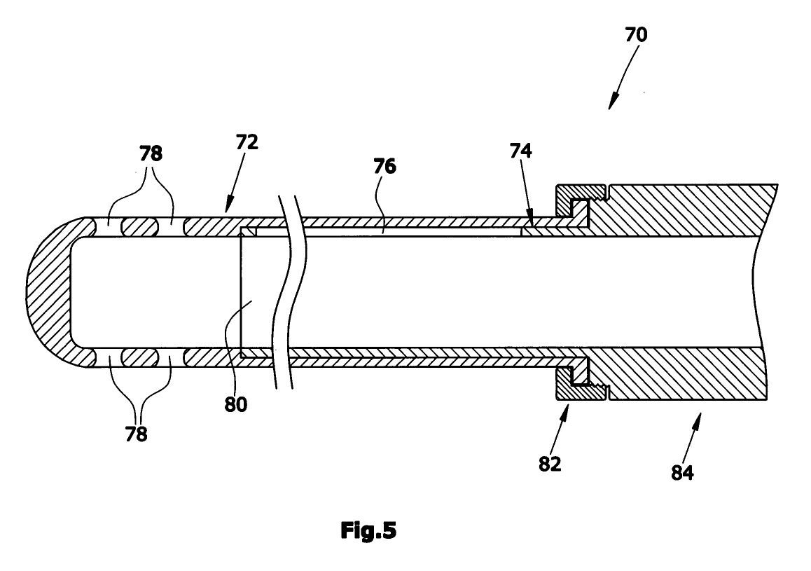

FIG. 5 is a third embodiment of a suction cannula in longitudinal section.

FIGS. 1-3 illustrate a suction cannula 10 in the form of a liposuction cannula for sucking body fat. Substantially, the suction cannula 10 is made up of a suction tube 12, a closure tube 14 acting as a cleaning aperture closure and a handle 16 that is able to be proximally screwed onto the suction tube 12 from outside. The suction tube 12, the closure tube 14 and the handle 16 are made of stainless steel.

The suction tube 12 has a circular cross section and is provided with a rounded cap 18 at its distal end. In the region of the tube jacket, several suction apertures 20 are provided at the distal end of the suction tube. It is also possible to provide a single suction aperture only which may also be arranged at the axial end of the suction tube 12.

In its central portion, the suction tube 12 comprises a longitudinal slot 24 as a cleaning aperture 22 which extends continuously almost to the handle 16. There may also be several cleaning apertures in the form of several shorter longitudinal slots which are arranged axially in series and separated from each other by material webs of the suction tube 12. This would result in a greater stability of the suction tube against radial stresses in circumferential direction.

At its proximal end, the suction tube 12 comprises a securing ridge 26 that is directed radially outward and serves to protect the closure tube 14 from twisting with respect to the suction tube 12. The securing ridge 26 is proximally followed by a male thread 28 cooperating with a corresponding female thread 30 of the handle 16. Together, the securing ridge 26, the suction tube male thread 28 and the handle female thread 30 form fastening elements fixing the suction tube 12 and the closure tube 14 with respect to each other as to all degrees of freedom.

The closure tube 14 also has a circular cross section and its inner diameter approximately corresponds to the outer diameter of the suction tube 12. The closure tube 14 comprises a continuous axial longitudinal slot 32 forming a cleaning aperture 34. Both the suction tube longitudinal slot and the closure tube longitudinal slot 32 have an opening angle of about 70°. The closed segment of the closure tube 14 forms a cleaning aperture closure 38 shown in the closing state in FIGS. 1-3 since it closes the cleaning aperture 22 of the suction tube 12 completely and liquid-tightly. At the proximal end of the closure tube 14, the suction tube 12 comprises a bead-like stop ring 40 with a ramp-like shape in longitudinal section and forming an axial stop for the closure tube 14 at the suction tube 12. The stop ring 40 is ramp-shaped and all of its edges are rounded to avoid tissue traumatizing during liposuction. This applies to all edges which might come into contact with tissue during a liposuction. If desired, the suction tube longitudinal slot 24 may also extend to the distal end of the suction tube 12. In this case, corresponding suction apertures have to be provided in the region of the suction tube longitudinal slot in any case if suction is desired in this region.

For cleaning and disinfecting, the suction cannula 10 is dismounted. This is effected by screwing the handle 16 off the suction tube 12 first. Subsequently, the closure tube 14 is pulled off the suction tube 12 in proximal-axial direction. Now, the suction tube 12 can be mechanically cleaned through the cleaning aperture 22 without any problem, e.g., by means of an appropriate brush and by the aid of a washing liquid which may both be introduced through the cleaning aperture 22.

The same applies to the cleaning of the closure tube 14 that can be cleaned mechanically in a thorough manner through its cleaning aperture 34 by, e.g., a brush and a washing liquid. In order to guarantee a good accessability to all regions for the purpose of cleaning and to avoid the adherence of residues in small niches, which cannot be caught or only hardly so during a mechanical cleaning, all regions which must be cleaned also have a generously rounded configuration, i.e., their radius is as large as possible.

After the mechanical cleaning, the suction tube 12, the closure tube 14 and the handle 16 are vapor-sterilized in the dismounted state. Only after the vapor-sterilization are the three mentioned parts reassembled to form an operative suction cannula 10. First, the closure tube 14 is pushed onto the suction tube 12 and subsequently, the handle 16 is screwed onto the suction tube 12. Thereby, the closure tube 14 is axially fixed on the suction tube 12 while the fixing in circumferential direction is effected by the securing ridge 26.

FIG. 4 illustrates a second embodiment of a suction cannula 50 where the closure tube 52 has a configuration similar to that of the suction tube 54, i.e., at its distal end, it is respectively closed completely by a cap.

The suction tube 54 comprises suction apertures 60 aligned with corresponding suction apertures 62 of the closure tube 52.

A suction tube-side nose 56 at the distal end of the suction tube 54 forms a twisting protection and engages into a corresponding inside recess 58 of the closure tube 52. Proximal to the suction apertures 60, 62, the suction cannula 50 has the same structure as the embodiment of a suction cannula 10 illustrated in the FIGS. 1-3.

In FIG. 5, a third embodiment of a suction cannula 70 is illustrated where the closure tube 72 is constructed as a disposable article and made of transparent plastic material. In principle, however, the plastic closure tube 72 may also be designed for being repeatedly used. The suction tube 74 is made of steel and can be used repeatedly. Therefore, the suction tube 74 has a cleaning aperture 76 configured as a longitudinal slot. In the present embodiment, radial suction apertures 78 are provided at the distal closed end of the closure tube 72. At its distal end, the suction tube 74 has an axial aperture 80 through which the biological tissue is sucked off. Moreover, the suction tube 74 serves the mechanical stabilization of the suction cannula 70 over approximately its entire length. Thus, a strong resilience of the suction cannula 70 with respect to bending moments is avoided, which cannot be granted over a certain length by a plastic closure tube 72 alone.

The disposable article closure tube 72 is fixed at the handle 84 of the suction tube 74 by a steel union nut 82. The disposable article closure tube may be fixed at the suction tube in many ways, i.e., by interlocking elements at the closure tube and the suction tube, by separate clamping levers etc.

The closure tube 72 is used only once and thereafter, it will not be sterilized but discarded. The closure tube 72 is producible in an injection-molding process at low costs. Since the closure tube is configured as an expendable article, it can be designed exclusively with respect to its suction functions. A good accessability for a mechanical cleaning and sterilization does not have to be considered in the construction of the closure tube. Particularly, it is no longer necessary to provide cleaning apertures at the closure tube 72 as well.

The cleaning apertures may also be exclusively provided in the suction tube, of course, similar to the suction tube 12 of FIG. 1, whereas the closure tube configured as an expendable article is configured similar to the closure tube 14 illustrated in FIG. 1 but does not have a cleaning aperture of its own.

1. A suction cannula (10) for sucking biological tissue from a human body, comprising
a suction tube (12) and at least one suction aperture (20),
characterized in
that the suction tube (12) comprises at least one cleaning aperture (12) and a cleaning aperture closure (38) that, in its closing state, closes the cleaning aperture (22) and, in its opening state, releases the cleaning aperture (22).

2. The suction cannula (10) of claim 1, characterized in that the cleaning aperture closure (38) is formed by a closure tube (14) seated on the outside of the suction tube (12) in the closing state and covering the cleaning aperture (22).

3. The suction cannula (70) of claim 2, characterized in that the closure tube (72) is an expendable article.

4. The suction cannula (70) of claim 3, characterized in that the suction tube (74) is made of metal and the closure tube (72) is made of steel.

5. The suction cannula (10) of one of claims 1-4, characterized in that the cleaning aperture (22) is formed by a longitudinal slot (24) in the suction tube (12).

6. The suction cannula (10) of claim 2 or 5, characterized in that the closure tube (14) comprises at least one cleaning aperture (34).

7. The suction cannula (10) of one of claims 1-6, characterized in that the suction aperture is provided in the closure tube (14).

8. The suction cannula (10) of one of claims 2-7, characterized in that the suction tube (12) and the closure tube (14) have cooperating fastening elements allocated thereto.

9. The suction cannula (10) of one of claims 1-8, characterized in that the cleaning aperture (22) is arranged proximal to the suction aperture (20).

10. The suction cannula (10) of one of claims 2-9, characterized in that the closure tube (72) has a transparent window or is transparent.
